(19) United States
(12) Patent Application Publication
(10) Pub. No.: US 2021/0162126 A1
(43) Pub. Date: Jun. 3, 2021

(54) Wearable Auto Injector

(71) Applicants: Daniel BARKHUFF, Albuquerque, NM (US); Justin T. BACA, Albuquerque, NM (US); Peter ZINK, Jamaica Plain, MA (US); Brandon WARRICK, Albuquerque, NM (US); STC.UNM, Albuquerque, NM (US)

(72) Inventors: Daniel Barkhuff, Albuquerque, NM (US); Justin T. Baca, Albuquerque, NM (US); Peter Zinc, Jamaica Plain, MA (US); Brandon Warrick, Albuquerque, NM (US)

(73) Assignee: STC.UNM, Albuquerque, NM (US)

(21) Appl. No.: 16/954,152

(22) PCT Filed: Oct. 11, 2018
(86) PCT No.: PCT/US2018/055506

§ 371 Date: Jun. 15, 2020

Related U.S. Application Data
(60) Provisional application No. 62/599,286, filed on Dec. 15, 2017.

(51) Int. Cl. A61M 5/172; A61M 5/142; A61M 5/20; A61M 5/315

(52) U.S. Cl. CPC A61M 5/1723; A61M 5/14248; A61M 5/20; A61M 5/31595; A61M 2005/14252; A61M 2230/63; A61M 2205/3375; A61M 2205/581; A61M 2205/18; A61M 2230/42; A61M 2205/3553

(57) ABSTRACT

An auto-injection system is having an injector, one or more sensors, and a controller, the controller activates the injector when the sensors detect a predetermined condition.

### RELATED APPLICATIONS

This application claims priority to U.S. Provisional Application No. 62/599,286 filed 15 Dec. 2017, which is incorporated in its entirety herewith.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Opioid overdose is the leading cause of mortality for individuals in the United States between the ages of 18 and 44. Opioid overdose is a significant public health emergency, resulting in hundreds of thousands of deaths in the past 15 years. Current naloxone therapies require a bystander or first responder to administer the medication.

### BRIEF SUMMARY OF THE INVENTION

In one embodiment, the present invention provides a system and method that administers a naloxone therapy in response to an overdose condition.

In another embodiment, the present invention provides a system and method that in response to an overdose condition couples with an injection system with a monitoring system that administers a therapy in response to a medical condition.

In another embodiment, the present invention provides a wearable naloxone delivery device that automatically delivers a naloxone therapy to reverse an otherwise lethal overdose.

In another embodiment, the present invention provides an auto-injection system comprising: an injector, one or more sensors, and a controller; the controller is in communication with the injector and the one or more sensors; the sensors are adapted to detect a predetermined condition, and the controller is adapted to cause the injector to actuate when a predetermined condition is detected.

In another embodiment, the present invention provides an auto-injection system further including an attachment means to affix the injector to a user.

In another embodiment, the present invention provides an auto-injection system including an attachment means that is an adjustable elastic strap, adhesive patch, or clip.

In another embodiment, the present invention provides an auto-injection system that includes a housing having an opening, a needle and a sterile membrane, the needle is located in the housing and the sterile membrane forms a penetrable seal over the opening.

In another embodiment, the present invention provides an auto-injection system wherein the predetermined condition is a respiratory rate.

In another embodiment, the present invention provides an auto-injection system further adapted to inject increasing doses of medication when no increase in respiratory rate is detected after a first dosage.

In another embodiment, the present invention provides an auto-injection system wherein the system has two positions, in a first position the needle is retracted in the housing; and in a second position, the needle extends through the membrane and out of the housing a sufficient distance to access the subcutaneous or intramuscular space of a patient.

In another embodiment, the present invention provides an auto-injection system wherein the needle is approximately 1 inch.

In another embodiment, the present invention provides an auto-injection system further including a pump connected to the needle, the pump is adapted to administer one or more doses of a preloaded medication.

In another embodiment, the present invention provides an auto-injection system wherein the pump is a syringe.

In another embodiment, the present invention provides an auto-injection system wherein the control mechanism is adapted to detect apnea in a patient.

In another embodiment, the present invention provides an auto-injection system wherein the sensors measure impedance changes by having a first sensor contact a user and a second sensor is located remotely from the first sensor and in contact with a user.

In another embodiment, the present invention provides an auto-injection system wherein at least one of the sensors is an acoustic sensor.

In another embodiment, the present invention provides an auto-injection system wherein at least one of the sensors plesmographically detects oxygenation.

In another embodiment, the present invention provides an auto-injection system wherein at least one of the sensors is a dermal sensor adapted to detect changes in lactate.

In another embodiment, the present invention provides an auto-injection system further including a motion sensor, the motion sensor is adapted to prevent inappropriate injections by being adapted to prevent activation of the injector if the sensor detects movement.

In another embodiment, the present invention provides an auto-injection system wherein the motion sensor is an accelerometer.

In another embodiment, the present invention provides an auto-injection system wherein the control mechanism is configured so that when the device detects a low respiratory rate, an audible alarm is activated.

In another embodiment, the present invention provides an auto-injection system further configured to inject naloxone to counteract an opiate overdose.

In another embodiment, the present invention provides an auto-injection system wherein the system is embedded in a garment.

In another embodiment, the present invention provides an auto-injection system wherein the system is embedded in an article of clothing.

In another embodiment, the present invention provides an auto-injection system wherein the system is embedded in a belt.

In another embodiment, the present invention provides an auto-injection system wherein the system is embedded in headgear.

In another embodiment, the present invention provides an auto-injection system wherein the system is embedded in a strap.

In another embodiment, the present invention provides an auto-injection system wherein the system is embedded in glasses.

In another embodiment, the present invention provides an auto-injection system wherein the system is used for mandated medications.

In another embodiment, the present invention provides an auto-injection system wherein the system is used with disabled patients.

In another embodiment, the present invention provides an auto-injection system wherein the system is as a safety measure.

In another embodiment, the present invention provides an auto-injection system wherein the system is used to administer anti-allergens, antidotes, and the analgesics.

In another embodiment, the present invention provides an auto-injection system wherein the needle is non-linear.

In another embodiment, the present invention provides an auto-injection system wherein the needle is curved.

In another embodiment, the present invention provides an auto-injection system wherein the needle is a straight needle connected to tubing that turns 90 degrees and is connect to a syringe or plunger.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates an embodiment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure, or system. Further, the terms and phrases used herein are not intended to be limiting, but rather to provide an understandable description of the invention.

In one embodiment the present provides an auto-injector worn close to the skin. The auto-injector monitors breathing while a patient is using opioids.

In other embodiments, the auto injector is configured to contain and administer a dose of naloxone or another medication that is administered if an overdose condition occurs. A method to monitor for an overdose condition is to monitor respiration.

In one preferred embodiment, the present invention provides a non-reusable, wearable, naloxone intramuscular auto-injector system 100 that activates, alarms, and fires when the user is in an overdose condition which may be detected when the user has become apneic. In a preferred embodiment, the invention may consist of three separate components all of which may be housed in a small housing 101. The components may include injector 105 that is configured to inject a medication. Monitoring a user may be accomplished by the use of one or more sensors 120 and 122. The sensors and injector may be operated by controller 130. The system may also include a power source 140.

In yet another embodiment, the present invention provides an auto-injector system that may be comprised of a wearable, adjustable elastic strap (not shown) to which the housing is attached. Similarly, in other embodiments, the device may be attached via a large adhesive (similar to a bandage) or by a clip that may be attached to clothing. This will allow the user to wear the auto-injector on their shoulder or across their chest, underneath clothing.

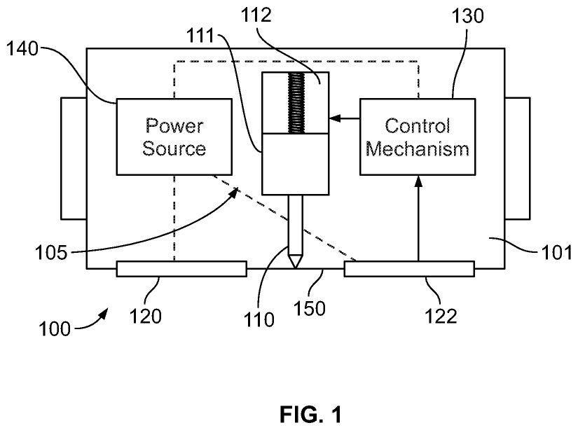

The housing of the auto-injector may be a plastic housing containing a needle 110, and a small syringe/injection device 112 approximately one inch in length which is sterile and injects through a sterile membrane 150 as shown in FIG. 1. In other embodiments, the present invention provides an auto-injector with the ability to inject increasing doses of a medication 111, such as naloxone, if the device detects no increase in respiratory rate after an earlier injection of the medication.

In yet other embodiments, the present invention provides a device which may have two positions of needle 110 in housing 101: one in which the needle and syringe are snugly fit inside of the housing, and a second position in which the needle extends outside of the housing a sufficient distance to access the subcutaneous or intramuscular space as shown in FIG. 1. The needle may be approximately 1 inch in length. Syringe 112 may have a plunger rod or other pumping mechanism which can inject one or more doses of the preloaded medication.

In other embodiments, the present invention provides a plunger that may be spring-loaded so that upon an order to fire, the device will rapidly advance the needle into position (protruding outside of the housing), followed one second later by a force applied to the plunger which will inject the medication.

In yet other embodiments, the impulse to activate and fire an injection may be instigated by an electronic detection device such as controller 130, situated in housing 101 which connects to one or more sensors and the injection device.

Controller 130 may be configured to detect a medical condition in a patient. In a preferred embodiment, the medical condition detected is apnea in a patient (defined as a respiratory rate of 6 or less although this will be adjustable).

In still further embodiments, the present invention provides a device that is capable of measuring impedance changes across two electrodes; one that contacts the user's skin at the site of the auto-injector housing, and another that is placed on the chest wall or an extremity. This second electrode may be incorporated into a watch or other wearable device. Other mechanisms of detecting decreased respiratory rate (or a surrogate or decreased respiratory rate) would be possible. These include an acoustic sensor, plesmographic detection of oxygenation, and dermal sensor capable of detecting changes in lactate.

This above-mentioned control mechanism may be configured to process the signal from the sensor and activate the injector when certain criteria such as bradypnea are met. The control mechanism could also include an accelerometer or other motion sensor to help prevent inappropriate injections. The control mechanism will not allow activation of the injector if it senses that the patient is still moving.

The control mechanism may be connected to the one or more sensors by wires, by wireless connection, or by an integrated wearable garment (e.g., an undershirt or vest that contains all parts of the device and any wires needed for the connections). Another feature of the control mechanism is that when the device detects a low respiratory rate, it may be configured to activate other features and components of the device which may be in the form of an audible alarm.

In yet other aspects, the present invention provides an apparatus and method covering the use of naloxone to counteract an opiate overdose. The present invention also provides an apparatus and method that may be used in the detection and treatment of sleep apnea. Another aspect of the present invention is that it provides an apparatus for injecting a drug based on biological information, thereby the drug, medication or treatment is automatically administered without the need for any interaction or decision by a user.

In other embodiments, the present invention may be used intramuscularly, and subcutaneously as well as by inhalation.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

1. An auto-injection system comprising:
an injector, one or more sensors, and a controller;
said controller in communication with said injector and said one or more sensors;
said sensors adapted to detect a predetermined condition; and
said controller adapted to cause said injector to actuate when a predetermined condition is detected.

2. The auto-injection system of claim 1 further including an attachment means to affix said injector to a user.

3. The auto-injection system of claim 2 wherein said attachment means is an adjustable elastic strap, adhesive patch, or clip.

4. The auto-injection system of claim 1 further including a housing having an opening, a needle and a sterile membrane, said needle located in said housing and said sterile membrane forming a penetrable seal over said opening.

5. The auto-injection system of claim 2 wherein said predetermined condition is a respiratory rate.

6. The auto-injection system of claim 1 further adapted to inject increasing doses of medication when no increase in respiratory rate is detected after a first dosage.

7. The auto-injection system of claim 4 wherein the system has two positions, in a first position said needle is retracted in said housing; and
in a second position, said needle extends through said membrane and out of said housing a sufficient distance to access the subcutaneous or intramuscular space of a patient.

8. The auto-injection system of claim 7 wherein the needle is approximately 1 inch in length.

9. The auto-injection system of claim 7 further including a pump connected to said needle, said pump adapted to administer one or more doses of a preloaded medication.

10. The auto-injection system of claim 9 wherein said pump is a syringe.

11. The auto-injection system of claim 1 wherein said control mechanism is adapted to detect apnea in a patient.

12. The auto-injection system of claim 1 wherein said sensors measure impedance changes by having a first sensor contact a user and a second sensor is located remotely from said first sensor and in contact with a user.

13. The auto-injection system of claim 1 wherein at least one of said sensors is an acoustic sensor.

14. The auto-injection system of claim 1 wherein at least one of said sensors plesmographically detects oxygenation.

15. The auto-injection system of claim 1 wherein at least one of said sensors is a dermal sensor adapted to detect changes in lactate.

16. The auto-injection system of claim 1 further including a motion sensor, said motion sensor is adapted to prevent inappropriate injections by being adapted to prevent activation of said injector if said sensor detects movement.

17. The auto-injection system of claim 16 wherein said motion sensor is an accelerometer.

18. The auto-injection system of claim 1 wherein said control mechanism is configured so that when the device detects a low respiratory rate, an audible alarm is activated.

19. The auto-injection system of claim 1 further configured to inject naloxone to counteract an opiate overdose.

20. The auto-injection system of claim 1 wherein said system is embedded in a garment.

21. (canceled)

22. (canceled)

23. (canceled)

24. (canceled)

25. (canceled)

26. (canceled)

27. (canceled)

28. (canceled)

29. (canceled)
